(19) United States
(12) Patent Application Publication
(10) Pub. No.: US 2011/0027211 A1
(43) Pub. Date: Feb. 3, 2011

(54) HAIR STYLING COMPOSITION

(75) Inventors: Sophie Viala, Koeln (DE); Sebastian Doerr, Duesseldorf (DE); Steffen Hofacker, Odenthal (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/934,849

(22) PCT Filed: Mar. 13, 2009
(86) PCT No.: PCT/EP09/01816

§ 371 Date: Sep. 27, 2010

(30) Foreign Application Priority Data
Mar. 26, 2008 (EP) 081553274.9

(51) Int. Cl. A61K 8/87; A61Q 5/06; C08G 18/28

(52) U.S. Cl. 424/70.17; 528/44; 528/68

(57) ABSTRACT

The invention relates to hair styling compositions containing special polyurethanes and to the use of the above-mentioned polyurethanes for producing hair styling compositions.

The present invention relates to hair setting compositions comprising special polyurethanes, and to the use of said polyurethanes for the preparation of hair setting compositions.

For shaping and stabilizing diverse hair styles, products are used which are known as hair setting compositions. Hair setting compositions are mostly in the form of setting mousses or hairsprays. Setting mousses and hairsprays differ little in their composition, but do differ in their application. Setting mousses are applied to damp hair as aids for modelling the hair style. In contrast to this, the hairsprays are sprayed onto dry ready-styled hair for fixing the hair style. Besides hairsprays and setting mousses, hair setting gels are also supplied.

In the case of hairsprays and setting mousses, the compositions for fixing or shaping the hair style are usually in the form of preparations which can be sprayed from aerosol containers, squeezy bottles or by pump, spray or foaming devices which consist of an alcoholic or aqueous-alcoholic solution of film-forming natural or synthetic polymers. These polymers can be selected from the group of nonionic, cationic, amphoteric or anionic polymers. In the case of hair setting gels, the preparations described above are adjusted to an acceptable viscosity using conventional thickeners.

During the formulation of cosmetic products, it is to be taken into consideration, as in the case of technical applications such as, for example, with paints, that, on account of the environmental regulations for controlling the emission of volatile organic compounds (VOCs) into the atmosphere, the proportion of propellants has to be reduced. Within the context of the present patent application, VOCs are understood as meaning organic, i.e. carbon-containing substances, which readily evaporate (are volatile) and/or are even in the form of a gas at low temperatures (e.g. room temperature (23° C.)). According to the invention, the VOCs include in particular organic substances with boiling points in the range from −90° to 300° C., in particular volatile hydrocarbons (e.g. propane, butane, isobutane, pentane, etc.), volatile ethers, such as dimethyl ether, volatile alcohols, such as methanol, ethanol, isopropanol and n-propanol and volatile amines. In particular, the proportion of VOCs through amines and organic solvents such as alcohols should be reduced in the cosmetic formulations, for example by replacing alcohols with water.

The film-forming polymers used in the prior art are preferably anionic or amphoteric polymers based on acrylates. The use of conventional acrylates in low-VOC hair setting formulations leads to problems with the stability of low-VOC compositions, such as sedimentation, separation etc. as is known to the person skilled in the art.

Furthermore, the conventional film-forming polymers exhibit low moisture and/or water resistance when the hair is in contact with rain or perspiration, and/or upon contact with moisture or under the influence of high atmospheric humidity, e.g. while bathing.

The use of polyurethanes in hair setting compositions is known. EP 1049446 A describes the use of certain polyurethanes in a hair care aerosol composition, where the valve, the opening and also the initial throughput of the aerosol composition are defined. Similarly, EP 1049443 A describes the use of certain polyurethanes in hair care aerosol formulations, where the formulations consist of 0.1 to 20% of polycondensate or polyurethanes and/or polyurea, 7.5 to 70% of organic solvent, 15 to 85% of propellant and 0.01 to 20% of at least one polyol. In the patents cited above, it was found that the sprayability of the hair aerosol is improved compared to the prior art.

EP 1652509 A describes a gel which comprises at least one polyurethane with a molecular weight between 400 000 and 5 000 000 g/mol and at least one thickener. In order to improve the water resistance, the applicant proposes the use of a polyurethane with a higher molecular weight compared with the polymers of the prior art. The advantages, described in the invention, of using a polyurethane with a high molecular weight were to be expected. However, the use of such a polymer in hairsprays and setting mousses is limited by the high molecular weight. The person skilled in the art is aware that the use of a film former with an excessively high molecular weight impairs the sprayability of the formulation.

Polyurethanes for hair setting compositions are also described in the following patents: EP 0751162, EP 0637600, FR 2743297, WO 9403510 and EP 0619111. WO 94/03510, EP 0619111 and EP 637600 describe polyurethanes of at least one diol containing acid or salt groups, in particular dimethylolpropanoic acid or N-methyldiethanolamine. EP 0751162 and FR 2743297 describe sequential polyurethane and/or polyurea polycondensates which consist of at least one polysiloxane block (FR 2743297) or of at least one polyurethane and/or polyurea block containing polysiloxane graft branches (EP 0751162), where the polyurethane block consists of at least one diol containing acid or salt groups, in particular dimethylolpropanoic acid.

EP 1457196 A describes a hair care composition for aerosol which consists of water, at least one organic solvent, at least one polyurethane and at least one propellant of dimethyl ether (DME) and at least one C3-4 hydrocarbon. The preferred polyurethanes consist of a divalent C2-C10 radical containing a carboxylic acid or sulfonic acid group, preferably dimethylolpropanoic acid. Luviset PUR (INCI name: Polyurethane-1) and Luviset Si PUR A (INCI name: Polyurethane-6=copolymer of dimethylolpropanoic acid, isophorone diisocyanate, neopentyl glycol, polyesterdiol and silicone diamine) are used for improving the sensory properties of hair setting sprays.

Similarly to EP 1457196, EP 789550 describes a composition comprising 10 to 60% by weight of DME, 39.9 to 89.9% of water, 0.1 to 15% by weight of polyurethanes and 0 to 5% by weight of alcohol having 1 to 4 hydrocarbon atoms. The polyurethanes used have a similar structure to that in EP 1457196 A.

DE 19541326 A describes water-soluble or water-dispersible polyurethanes of:
a) a water-soluble or -dispersible urethane prepolymer with terminal isocyanate groups and
b) at least one primary or secondary amine which has at least one ionogenic or ionic group, and the salts thereof.

The urethane prepolymer is a water-soluble or water-dispersible polyurethane with terminal isocyanate groups which has ionogenic or ionic groups bonded to the polymer chain. The ionogenic or ionic groups can comprise carboxylic acid groups and/or sulfonic acid groups and/or nitrogen-containing groups, in particular dimethylolpropanoic acid. This results in a comparatively high hydrophilicity of the urethane prepolymer.

The primary or secondary amines react with the terminal isocyanate groups of the urethane prepolymer and are bonded to the polyurethane via a urea group. The amines are particularly preferably taurine, N-(1,1-dimethyl-2-hydroxyethyl)-3-amino-2-hydroxypropane sulfonic acid or 4-aminobenzene sulfonic acid. The use of said polyurethanes as aids in cosmetics, in particular as hair setting compositions, is mentioned. Compared to other polyurethane polymers of the prior art, the polyurethanes are easier to wash out in experiments on artificial model heads.

However, the patents cited above do not mention important properties of the film former such as the resistance to moisture or water. The polyurethanes of the prior art are hydrophilic especially through use of 2-2-hydroxymethyl-substituted carboxylic acids, preferably dimethylolpropanoic acid. By neutralizing the carboxyl groups on the main chain with amines or alkalis, the incorporation of the polyurethane into a formulation with a low VOC becomes possible. The more carboxylate groups present, the more hydrophilic the polymer and thus the better it dissolves in a solvent system which comprises a high proportion of water. Formulations with a low content of VOC are possible with the polyurethanes described above, but at the expense of important properties such as, in particular, curl hold at high humidity. This is because the large number of acid groups in the polymer chain accelerates the collapse of the hair style.

Furthermore, in the currently known aqueous systems based on polyurethane, besides solvents, volatile amines are often also present which are used as neutralizers. From toxicological points of view, these amines may be critical for such an application close to the body.

A further disadvantage of the systems according to the prior art is the large amount of the corresponding film former which has to be added to achieve the desired effects. This restricts the freedom when creating the formulation and can, moreover, lead to increased costs.

The object of the present invention is therefore to provide a low-VOC hair setting composition which has a very high moisture resistance even at a low use concentration of the polyurethanes and leads to very good curl hold.

The present invention thus provides in particular low-VOC hair setting formulations which have excellent curl hold even at high humidity and at low use concentrations of the polyurethane, compared to the hair setting formulations from the prior art.

It has surprisingly been found that special polyurethanes, in particular aqueous polyurethane dispersions comprising them, which are prepared from water-insoluble, non-water-dispersible, isocyanate-functional prepolymers are particularly well suited for achieving the object.

The present invention thus provides a hair setting composition comprising at least one polyurethane obtainable by reacting one or more water-insoluble, non-water-dispersible, isocyanate-functional polyurethane prepolymers A) with one or more amino-functional compounds B).

Within the context of the invention, the term “water-insoluble, non-water-dispersible polyurethane prepolymer” means in particular that the solubility in water of the prepolymer used according to the invention at 23° C. is less than 10 g/litre, more preferably less than 5 g/litre, and the prepolymer does not produce a sedimentation-stable dispersion in water, in particular deionized water, at 23°. In other words, the prepolymer settles out upon any attempt to disperse it in water.

Preferably, the polyurethane prepolymer A) used according to the invention has terminal isocyanate groups, i.e. the isocyanate groups are at the chain ends of the prepolymer. All of the chain ends of a prepolymer particularly preferably have isocyanate groups.

Furthermore, the polyurethane prepolymer A) used according to the invention preferably has essentially neither ionic nor ionogenic groups, i.e. the content of ionic and ionogenic groups is expediently below 15 milliequivalents per 100 g of polyurethane prepolymer A), preferably below 5 milliequivalents, particularly preferably below 1 milliequivalent and very particularly preferably below 0.1 milliequivalent per 100 g of polyurethane prepolymer A).

The amino-functional compounds B) are preferably selected from primary and/or secondary amines and/or diamines. In particular, the amino-functional compounds B) include at least one diamine. The amino-functional compounds B) are preferably selected from amino-functional compounds B2), which have ionic or ionogenic groups, and amino-functional compounds B1), which have no ionic or ionogenic group.

In a particularly preferred embodiment of the invention, the amino-functional compounds B) include at least one amino-functional compound B2) which has ionic and/or ionogenic (ion-forming) groups. The ionic and/or ionogenic group used is particularly preferably the sulfonate or the sulfonic acid group, yet more preferably the sodium sulfonate group.

In a further preferred embodiment of the invention, the amino-functional compounds B) include both amino-functional compounds B2) which have ionic and/or ionogenic group, and also amino-functional compounds B1) which have no ionic or ionogenic group.

Accordingly, polyurethanes within the context of the invention are polymeric compounds which have at least two, preferably at least three, repeat units containing urethane groups:

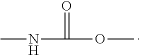

According to the invention, also included are those polyurethanes which, as a result of the preparation, also have repeat units containing urea groups:

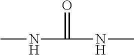

as are formed in particular in the reaction of the isocyanate-terminated prepolymers A) with the amino-functional compounds B).

The hair setting compositions according to the invention are in particular water-containing, i.e. aqueous, compositions in which the polyurethane is present in dispersed form, i.e. in essentially non-dissolved form. Besides other liquid media which may be present if desired, such as, for example, solvents, water generally forms the main constituent (>50% by weight) of the dispersion media, based on the total amount of the liquid dispersion media in the cosmetic compositions according to the invention, in some cases also the only liquid dispersion medium.

The hair setting compositions according to the invention preferably have a content of volatile organic compounds (VOCs) of less than 80% by weight, more preferably of less than 55% by weight, even more preferably of less than 40% by weight, based on the hair setting composition.

The aqueous polyurethane dispersions used for the preparation of the hair setting compositions according to the invention preferably have a content of volatile organic compounds (VOCs) of less than 10% by weight, more preferably of less than 3% by weight, even more preferably of less than 1% by weight, based on the aqueous polyurethane dispersion.

The content of volatile organic compounds (VOCs) is determined within the context of the present invention in particular by gas chromatographic analysis.

The non-water-soluble and non-water-dispersible, isocyanate-functional polyurethane polymers used according to the invention have essentially neither ionic nor ionogenic groups. The insolubility in water and/or lack of dispersibility in water refers to deionized water without the addition of surfactants. Within the context of the present invention this means that the proportion of ionic and/or ionogenic (ion-forming) groups, such as, in particular, anionic groups, such as carboxylate or sulfonate, or of cationic groups is less than 15 milliequivalents per 100 g of polyurethane prepolymer A), preferably less than 5 milliequivalents, particularly preferably less than 1 milliequivalent and very particularly preferably less than 0.1 milliequivalent per 100 g of polyurethane prepolymer A).

In the case of acidic ionic and/or ionogenic groups, the acid number of the prepolymer is expediently below 30 mg of KOH/g of prepolymer, preferably below 10 mg of KOH/g of prepolymer. The acid number indicates the mass of potassium hydroxide in mg which is required to neutralize 1 g of the sample under investigation (measurement in accordance with DIN EN ISO 211). The neutralized acids, i.e. the corresponding salts, naturally have no acid number or a reduced acid number. According to the invention, the acid number of the corresponding free acid is decisive here.

The prepolymers A) used for the preparation of the polyurethanes are preferably obtainable by reacting one or more polyols selected from the group which consists of polyether polyols, polycarbonate polyols, polyether polycarbonate polyols and/or polyester polyols, and polyisocyanates, as is explained in more detail below.

The polyurethanes present in the hair setting compositions according to the invention accordingly comprise, via the prepolymer A), preferably at least one sequence selected from the group which consists of: polyether, polycarbonate, polyether-polycarbonate and polyester sequences. According to the invention, this means in particular that the polyurethanes contain repeat units containing ether groups and/or carbonate groups or ester groups. The polyurethanes can contain, for example, exclusively polyether sequences or exclusively polycarbonate sequences or exclusively polyester sequences. However, they can also have both polyether and polycarbonate sequences, as are formed, for example, during the preparation of polycarbonate polyols using polyetherdiols, as is described in more detail below. In addition, they can have polyether-polycarbonate sequences which arise from the use of polyether-polycarbonate polyols, as described in more detail below.

Particularly preferred polyurethanes are obtained using polymeric polyether polyols and/or polymeric polycarbonate polyols and/or polyether-polycarbonate polyols or polyester polyols, each of which have number-average molecular weights of preferably about 400 to about 6000 g/mol (here and in the case of the molecular weight data below, determined by gel permeation chromatography relative to polystyrene standard in tetrahydrofuran at 23° C.). Their use during the preparation of the polyurethanes or polyurethane prepolymers leads, as a result of the reaction with polyisocyanates, to the formation of corresponding polyether and/or polycarbonate and/or polyether-polycarbonate sequences or polyester sequences in the polyurethanes with a corresponding molecular weight of these sequences. According to the invention, particular preference is given to polyurethanes which are obtained from polymeric polyetherdiols and/or polymeric polycarbonatediols and/or polyether-polycarbonate polyols or polyester polyols with a linear structure.

The polyurethanes according to the invention are preferably essentially linear molecules, but may also be branched, which is less preferred.

The number-average molecular weight of the polyurethanes preferably used according to the invention is, for example, from about 1000 to 200 000, preferably from 5000 to 150 000. Molecular weights above 200 000 can be disadvantageous under certain circumstances since the hair setting compositions are sometimes difficult to wash out.

The polyurethanes present in the hair setting compositions according to the invention are added to the specified compositions in particular in the form of aqueous dispersions.

Preferred polyurethanes or polyurethane dispersions to be used according to the invention are obtainable by preparing

A) isocyanate-functional prepolymers of
A1) organic polyisocyanates,
A2) polymeric polyols, preferably with number-average molecular weights of from 400 to 8000 g/mol (here and in the case of the molecular weight data below, determined by gel permeation chromatography relative to polystyrene standard in tetrahydrofuran at 23° C.), more preferably 400 to 6000 g/mol and particularly preferably from 600 to 3000 g/mol, and OH functionalities of preferably 1.5 to 6, more preferably 1.8 to 3, particularly preferably from 1.9 to 2.1,
A3) optionally hydroxy-functional compounds with molecular weights of preferably 62 to 399 g/mol, and
A4) optionally nonionic hydrophilizing agents,
and
   B) then reacting some or all of their free NCO groups
with one or more amino-functional compounds B), such as primary and/or secondary amines and/or diamines.

The polyurethanes used according to the invention are preferably dispersed in water before, during or after step B).

The reaction with a diamine or two or more diamines in step B) particularly preferably takes place with chain extension. In this connection, monofunctional amines can additionally be added as chain terminators to control the molecular weight.

As component B), in particular amines can be used which have no ionic or ionogenic, such as anionically hydrophilizing groups (component B1 below)) and it is possible to use amines which have ionic or ionogenic, such as, in particular, anionically hydrophilizing groups (component B2 below)).

Preferably, in step B) of the reaction of the prepolymer, a mixture of component B1) and component B2) is reacted. By using component B1) it is possible to build up a high molar mass without the viscosity of the previously prepared isocyanate-functional prepolymer increasing to a degree which would be an obstacle to processing. By using the combination of components B1) and B2) it is possible to achieve an optimum balance between hydrophilicity and chain length and thus good substantivity without “build-up” effects arising.

The polyurethanes used according to the invention preferably have anionic groups, preferably sulfonate groups. These anionic groups are introduced into the polyurethanes used according to the invention via the amine component B2) reacted in step B). The polyurethanes used according to the invention optionally additionally have nonionic components for hydrophilization. Exclusively sulfonate groups are particularly preferably present in the polyurethanes used according to the invention for the hydrophilization; these are introduced into the polyurethane via corresponding diamines as component B2).

In order to achieve a good sedimentation stability, the number-average particle size of the special polyurethane dispersions is preferably less than 750 nm, particularly preferably less than 500 nm, determined by means of laser correlation spectroscopy following dilution with deionized water (instrument: Malvern Zetasizer 1000, Malver Inst. Limited).

The solids content of the polyurethane dispersions which is preferably used for preparing the hair setting composition of the invention is generally 10 to 70% by weight, preferably 30 to 65% by weight, particularly preferably 40 to 60% by weight. The solids contents are ascertained by heating a weighed sample at 125° C. to constant weight. At constant weight, the solid-body content is calculated by reweighing the sample.

Preferably, these polyurethane dispersions have less than 5% by weight, particularly preferably less than 0.2% by weight, based on the mass of the dispersions, of unbonded organic amines. The content in the hair setting compositions is correspondingly yet lower.

Suitable polyisocyanates of component A1) are in particular the aliphatic, aromatic or cycloaliphatic polyisocyanates with an NCO functionality of greater than or equal to 2 known per se to the person skilled in the art.

Examples of such suitable polyisocyanates are 1,4-butylene diisocyanate, 1,6-hexamethylene diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any desired isomer content, 1,4-cyclohexylene diisocyanate, 4-isocyanatomethyl-1,8-octane diisocyanate (nonane triisocyanate), 1,4-phenylene diisocyanate, 2,4- and/or 2,6-tolylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), and alkyl 2,6-diisocyanatohexanoates (lysine diisocyanates) with C1-C8-alkyl groups.

Besides the aforementioned polyisocyanates, it is also possible to use modified diisocyanates which have a functionality of ≧2 with uretdione, isocyanurate, urethane, allophanate, biuret, iminooxadiazinedione or oxadiazinetrione structure, and also mixtures of these proportionately.

They are preferably polyisocyanates or polyisocyanate mixtures of the type specified above with exclusively aliphatically or cycloaliphatically bonded isocyanate groups or mixtures of these and an average NCO functionality of the mixture of from 2 to 4, preferably 2 to 2.6 and particularly preferably 2 to 2.4, very particularly preferably 2.

Hexamethylene diisocyanate, isophorone diisocyanate or the isomeric bis(4,4′-isocyanatocyclohexyl)methanes, and mixtures of the aforementioned diisocyanates are particularly preferably used in A1).

In A2), polymeric polyols with a number-average molecular weight Mₙ of preferably 400 to 8000 g/mol, more preferably from 400 to 6000 g/mol and particularly preferably from 600 to 3000 g/mol are used. These preferably have an OH functionality of from 1.5 to 6, particularly preferably from 1.8 to 3, very particularly preferably from 1.9 to 2.1.

The expression “polymeric” polyols means here in particular that the specified polyols have at least two, more preferably at least three, repeat units joined together.

Such polymeric polyols are the polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols known per se in polyurethane coating technology. These can be used in A2) individually or in any desired mixtures with one another.

The preferably used polyester polyols are the polycondensates known per se of di- and optionally tri- and tetraols and di- and optionally tri- and tetracarboxylic acids or hydroxycarboxylic acids or lactones. Instead of the free polycarboxylic acids, it is also possible to use the corresponding polycarboxylic acid anhydrides or corresponding polycarboxylic acid esters of lower alcohols for the preparation of the polyesters.

Examples of suitable diols are ethylene glycol, butylene glycol, diethylene glycol, triethylene glycol, polyalkylene glycols, such as polyethylene glycol, also 1,2-propanediol, 1,3-propanediol, butanediol(1,3), butanediol(1,4), hexanediol(1,6) and isomers, neopentyl glycol or hydroxypivalic neopentyl glycol ester, where hexanediol(1,6) and isomers, butanediol(1,4), neopentyl glycol and hydroxypivalic neopentyl glycol ester are preferred. In addition, polyols such as trimethylol propane, glycerol, erythritol, pentaerythritol, trimethylol benzene or trishydroxyethyl isocyanurate can also be used.

Dicarboxylic acids which can be used are phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. The corresponding anhydrides may also be used as acid source.

If the average functionality of the polyol to be esterified is >than 2, monocarboxylic acids, such as benzoic acid and hexanecarboxylic acid, can additionally also be co-used.

Preferred acids are aliphatic or aromatic acids of the type specified above. Particular preference is given to adipic acid, isophthalic acid and phthalic acid.

Hydroxycarboxylic acids which can be co-used as reactants in the preparation of a polyester polyol with terminal hydroxyl groups are, for example, hydroxycaproic acid, hydroxybutyric acid, hydroxydecanoic acid, hydroxystearic acid and the like. Suitable lactones are caprolactone, butyrolactone and homologs. Preference is given to caprolactone.

According to the invention, particularly preferred components A2) for the preparation of the polyurethanes are polyester polyols with a number-average molecular weight of from 600 to 3000 g/mol, in particular aliphatic polyester polyols based on aliphatic carboxylic acids and aliphatic polyols, in particular based on adipic acid and aliphatic alcohols, such as hexanediol and/or neopentyl glycol.

Polycarbonates having hydroxyl groups, preferably polycarbonatediols, with number-average molecular weights Mₙ of from preferably 400 to 8000 g/mol, preferably 600 to 3000 g/mol can likewise be used as component A2). These are obtainable by reacting carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentanediol-1,3, dipropylene glycol, polypropylene glycols, dibutylene glycol, polybutylene glycols, bisphenol A and lactone-modified diols of the type specified above.

Preferably, the diol component comprises 40 to 100% by weight of hexanediol, preference being given to 1,6-hexanediol and/or hexanediol derivatives. Such hexanediol derivatives are based on hexanediol and, besides terminal OH groups, have ester or ether groups. Such derivatives are obtainable by reacting hexanediol with excess caprolactone or by etherifying hexanediol with itself to give the di- or trihexylene glycol.

Instead of or in addition to the pure polycarbonatediols, it is also possible to use polyether-polycarbonatediols in A2).

Polycarbonates having hydroxyl groups preferably have a linear structure.

Polyether polyols can likewise be used as component A2).

For example, the polytetramethylene glycol polyethers known per se in polyurethane chemistry, as are obtainable through polymerization of tetrahydrofuran by means of cationic ring opening, are particularly suitable.

Likewise suitable polyether polyols are the addition products, known per se, of styrene oxide, ethylene oxide, propylene oxide, butylene oxide and/or epichlorohydrin onto di- or polyfunctional starter molecules. Thus, in particular polyalkylene glycols, such as polyethylene glycols, polypropylene glycols and/or polybutylene glycols, can be used, in particular those with the preferred molecular weights specified above.

Suitable starter molecules which can be used are all compounds known according to the prior art, such as, for example, water, butyl diglycol, glycerol, diethylene glycol, trimethylolpropane, propylene glycol, sorbitol, ethylenediamine, triethanolamine, 1,4-butanediol.

Particularly preferred components in A2) are polytetramethylene glycol polyethers and polycarbonate polyols and mixtures thereof and particularly preferably polytetramethylene glycol polyethers.

In preferred embodiments of the invention, component A2) is accordingly:
mixtures comprising at least one polyether polyol and at least one polycarbonate polyol,
mixtures comprising more than one polyether polyol, or a mixture of two or more polyether polyols with different molecular weights, which are in particular poly(tetramethylene glycol) polyether polyols (such as HO—(CH₂—CH₂—CH₂—CH₂—O)ₙ—H),
mixtures comprising more than one polyether polyol and at least one polycarbonate polyol, and also
particularly preferably polyester polyols with a number-average molecular weight of from 600 to 3000 g/mol, in particular aliphatic polyester polyols based on aliphatic carboxylic acids and aliphatic polyols, in particular based on adipic acid and aliphatic alcohols, such as hexanediol and/or neopentyl glycol,
   where component A), according to the definition, has essentially neither ionic nor ionogenic groups.

As component A3), polyols, in particular nonpolymeric polyols, of the specified preferred molecular weight range from 62 to 399 mol/g with up to 20 carbon atoms, such as ethylene glycol, diethylene glycol, triethylene glycol, 1,2-propanediol, 1,3-propanediol, 1,4-butanediol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol, and any mixtures thereof can be used as desired.

Also suitable are ester diols of the specified molecular weight range, such as α-hydroxybutyl ε-hydroxycaproic acid ester, ω-hydroxyhexyl γ-hydroxybutyric acid ester, adipic acid (β-hydroxyethyl) ester or terephthalic acid bis(β-hydroxyethyl) ester.

In addition, as component A3), it is also possible to use monofunctional isocyanate-reactive hydroxyl-group-containing compounds. Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

In one preferred embodiment of the invention, the polyurethane used according to the invention comprises less than about 10% by weight of component A3), preferably less than 5% by weight of component A3), in each case based on the total mass of the polyurethane, yet more preferably component A3) is not used for the preparation of the polyurethane.

To prepare the polyurethanes used according to the invention, one or more in particular isocyanate-reactive nonionic hydrophilizing agents are optionally used as component A4). The hydrophilizing agents used as component A4) are in particular different from components A2) and A3).

Suitable nonionically hydrophilizing compounds as component A4) are, for example, polyoxyalkylene ethers which have isocyanate-reactive groups, such as hydroxy, amino or thiol groups. Preference is given to monohydroxy-functional polyalkylene oxide polyether alcohols having, on statistical average, 5 to 70, preferably 7 to 55, ethylene oxide units per molecule, as are accessible in a manner known per se by alkoxylation of suitable starter molecules (e.g. in Ullmanns Encyclopädie der technischen Chemie [Ullmanns encyclopaedia of industrial chemistry], 4th edition, Volume 19, Verlag Chemie, Weinheim pp. 31-38). These are either pure polyethylene oxide ethers or mixed polyalkylene oxide ethers, where they contain at least 30 mol %, preferably at least 40 mol %, ethylene oxide units, based on all of the alkylene oxide units present.

Particularly preferred nonionic compounds are monofunctional mixed polyalkylene oxide polyethers which have 40 to 100 mol % ethylene oxide units and 0 to 60 mol % propylene oxide units.

Suitable starter molecules for such nonionic hydrophilizing agents are in particular saturated monoalcohols, such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, such as, for example, diethylene glycol monobutyl ether, unsaturated alcohols, such as allyl alcohol, 1,1-dimethylallyl alcohol or oleyl alcohol, aromatic alcohols, such as phenol, the isomeric cresols or methoxyphenols, araliphatic alcohols, such as benzyl alcohol, anisyl alcohol or cinnamyl alcohol, secondary monoamines, such as dimethylamine, diethylamine, dipropylamine, diisopropylamine, dibutylamine, bis(2-ethylhexyl)amine, N-methyl- and N-ethylcyclohexylamine or dicyclohexylamine, and also heterocyclic secondary amines, such as morpholine, pyrrolidine, piperidine or 1H-pyrazole. Preferred starter molecules are saturated monoalcohols of the type specified above. Particular preference is given to using diethylene glycol monobutyl ether or n-butanol as starter molecules.

Alkylene oxides suitable for the alkoxylation reaction are in particular ethylene oxide and propylene oxide, which can be used in the alkoxylation reaction in any desired order or else in a mixture.

Component B) is preferably selected from primary or secondary amine and/or diamines. It includes in particular diamines.

As component B) it is possible to use in particular amines which have no ionic or ionogenic, such as anionically hydrophilizing groups (component B1) below), and it is possible to use amines which have ionic or ionogenic, such as, in particular, anionically hydrophilizing groups (component B2) below). Preferably, in step B) of the reaction of the prepolymer, a mixture of component B1) and of component B2) is reacted.

For example, organic di- or polyamines, such as, for example, 1,2-ethylenediamine, 1,2- and 1,3-diaminopropane, 1,4-diaminobutane, 1,6-diaminohexane, isophorone-diamine, isomer mixture of 2,2,4- and 2,4,4-trimethylhexamethylenediamine, 2-methylpentamethylenediamine, diethylenetriamine, 4,4-diaminodicyclohexylmethane, hydrazine hydrate, and/or dimethylethylenediamine, can be used as component B1).

Moreover, compounds which, besides a primary amino group, also have secondary amino groups or, besides an amino group (primary or secondary), also have OH groups, can also be used as component B1). Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines, such as N-aminoethylethanolamine, ethanolamine, 3-aminopropanol, neopentanolamine.

In addition, monofunctional isocyanate-reactive amine compounds can also be used as component B1), such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and suitable substituted derivatives thereof, amidoamines of diprimary amines and monocarboxylic acids, monoketime of diprimary amines, primary/tertiary amines, such as N,N-dimethylaminopropylamine.

As component B1), preference is given to using 1,2-ethylenediamine, bis(4-aminocyclohexyl)methane, 1,4-diaminobutane, isophoronediamine, ethanolamine, diethanolamine and diethylenetriamine.

Component B) particularly preferably includes at least one component B2). Suitable anionically hydrophilizing compounds as component B2) preferably contain a sulfonic acid or sulfonate group, particularly preferably a sodium sulfonate group. Suitable anionically hydrophilizing compounds as component B2) are, in particular, the alkali metal salts of mono- and diaminosulfonic acids. Examples of such anionic hydrophilizing agents are salts of 2-(2-aminoethylamino)ethanesulfonic acid, ethylenediamine-propyl- or -butylsulfonic acid, 1,2- or 1,3-propylenediamine-1′-ethylsulfonic acid or taurine. Furthermore, the salt of cyclohexylaminopropanesulfonic acid (CAPS) from WO-A 01/88006 can be used as anionic hydrophilizing agent.

Particularly preferred anionic hydrophilizing agents B2) are those which contain sulfonate groups as ionic groups and two amino groups, such as the salts of 2-(2-aminoethylamino)ethylsulfonic acid and 1,3-propylenediamine-β-ethylsulfonic acid.

The polyurethanes used according to the invention particularly preferably comprising at least one sulfonate group.

Optionally, the anionic group in component B2) may also be a carboxylate or carboxylic acid group. Component B2) is then preferably selected from diaminocarboxylic acids. However, this embodiment is less preferred since carboxylic-acid-based components B2) have to be used in higher concentrations.

For the hydrophilization, it is also possible to use mixtures of anionic hydrophilizing agents B2) and nonionic hydrophilizing agents A4).

In a preferred embodiment for the preparation of the special polyurethane dispersions, components A1) to A4) and B1) to B2) are used in the following amounts, the individual amounts always adding up to 100% by weight:

5 to 40% by weight of component A1),
55 to 90% by weight of A2),
0.5 to 20% by weight sum of components A3) and/or B1),
0.1 to 25% by weight sum of components A4) and/or B2), where, based on the total amounts of components A1) to A4) and B1) to B2), particularly preferably 0.1 to 5% by weight of anionic or potentially anionic hydrophilizing agents B2) are used.

In a particularly preferred embodiment for the preparation of the special polyurethane dispersions, components A1) to A4) and B1) to B2) are used in the following amounts, the individual amounts always adding up to 100% by weight:

5 to 35% by weight of component A1),
60 to 90% by weight of A2),
0.5 to 15% by weight sum of components A3) and/or B1),
0.1 to 15% by weight sum of components A4) and/or B2), where, based on the total amounts of components A1) to A4) and B1) to B2), particularly preferably 0.2 to 4% by weight of anionic or potentially anionic hydrophilizing agents B2) are used.

In a very particularly preferred embodiment for the preparation of the special polyurethane dispersions, components A1) to A4) and B1) to B2) are used in the following amounts, the individual amounts always adding up to 100% by weight:

10 to 30% by weight of component A1),
65 to 85% by weight of A2),
0.5 to 14% by weight sum of components A3 and/or B1),
0.1 to 13.5% by weight sum of components A4) and/or B2), where, based on the total amounts of components A1) to A4) and B1) to B2), particularly preferably 0.5 to 3.0% by weight of anionic or potentially anionic hydrophilizing agents from B2) are used.

The preparation of the polyurethane dispersions can be carried out in one or more stage(s) in homogeneous phase or, in the case of multistage reaction, sometimes in disperse phase. Following complete or partial polyaddition from A1) to A4), a dispersion, emulsification or dissolution step preferably takes place. Afterwards, a further polyaddition or modification optionally takes place in the disperse phase.

In this connection, all of the methods known from the prior art, such as, for example, prepolymer mixing process, acetone process or melt dispersion process, can be used. Preference is given to using the acetone process.

For the preparation in accordance with the acetone process, constituents A2) to A4) and the polyisocyanate component A1) for the preparation of an isocyanate-functional polyurethane prepolymer are usually initially introduced in their entirety or in part and optionally diluted with a solvent which is miscible with water but inert towards isocyanate groups, and heated to temperatures in the range from 50 to 120° C. To increase the rate of the isocyanate addition reaction, the catalysts known in polyurethane chemistry can be used.

Suitable solvents are the customary aliphatic, keto-functional solvents such as acetone, 2-butanone, which can be added not only at the start of the preparation, but optionally in parts also later on. Preference is given to acetone and 2-butanone, and particular preference is given to acetone. The addition of other solvents without isocyanate-reactive groups is also possible, but not preferred.

Any constituents of A1) to A4) not added at the start of the reaction are then metered in.

During the preparation of the polyurethane prepolymer from A1) to A4), the quantitative ratio of isocyanate groups to isocyanate-reactive groups is generally 1.05 to 3.5, preferably 1.1 to 3.0, particularly preferably 1.1 to 2.5.

The reaction of components A1) to A4) to give the prepolymer takes place partially or completely, but preferably completely. Polyurethane prepolymers which contain free isocyanate groups are thus obtained without a diluent or in solution.

In the neutralization step for the partial or complete conversion of potentially anionic groups to anionic groups, bases such as tertiary amines, e.g. trialkylamines having 1 to 12, preferably 1 to 6, carbon atoms, particularly preferably 2 to 3 carbon atoms in each alkyl radical or very particularly preferably alkali metal bases such as the corresponding hydroxides are used.

The use of organic amines is not preferred.

Neutralizing agents which can be used are preferably inorganic bases, such as aqueous ammonia solution or sodium hydroxide or potassium hydroxide.

Preference is given to sodium hydroxide and potassium hydroxide.

The quantitative amount of the bases is 50 and 125 mol %, preferably between 70 and 100 mol % of the quantitative amount of the acid groups to be neutralized. The neutralization can also take place at the same time as the dispersion by the dispersion water already comprising the neutralizing agent.

Afterwards, in a further process step, in cases where it has still not happened or has only happened partially, the resulting prepolymer is dissolved with the help of aliphatic ketones such as acetone or 2-butanone.

The reaction of components A1) to A4) to give the prepolymer takes place partially or completely, but preferably completely. In this way, polyurethane prepolymers which contain free isocyanate groups are obtained without a diluent or in solution.

During the chain extension in stage B), NH₂- and/or NH-functional components are reacted with the remaining isocyanate groups of the prepolymer. Preferably, the chain extension/termination is carried out prior to the dispersion in water.

Suitable components B) for the chain extension are, in particular, organic di- or polyamines B1), such as, for example, ethylenediamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, diaminodicyclohexylmethane and/or dimethylethylenediamine.

Moreover, it is also possible to use compounds B1) which, besides a primary amino group, also have secondary amino groups or, besides an amino group (primary or secondary), also have OH groups. Examples thereof are primary/secondary amines, such as diethanolamine, 3-amino-1-methylaminopropane, 3-amino-1-ethylaminopropane, 3-amino-1-cyclohexylaminopropane, 3-amino-1-methylaminobutane, alkanolamines, such as N-aminoethylethanolamine, ethanolamine, 3-aminopropanol, neopentanolamine be used for the chain extension and/or termination.

For the chain termination, use is usually made of amines B1) having a group which is reactive towards isocyanates, such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, and suitable substituted derivatives thereof, amidoamines of diprimary amines and monocarboxylic acids, monoketime of diprimary amines, primary/tertiary amines, such as N,N-dimethylaminopropylamine.

If anionic hydrophilizing agents corresponding to the definition of B2) with NH₂ or NH groups are used for the chain extension, the chain extension of the prepolymers preferably takes place before the dispersion.

The degree of chain extension, i.e. the equivalent ratio of NCO-reactive groups of the compounds used for the chain extension and chain termination to free NCO groups of the prepolymer is generally between 40 and 150%, preferably between 50 and 110%, particularly preferably between 60 and 100%.

The aminic components B1) and B2) can optionally be used in water- or solvent-diluted form in the process according to the invention individually or in mixtures, with any order of the addition being possible in principle.

If water or organic solvents are co-used as diluents, then the diluent content in the component used in B) for the chain extension is preferably 40 to 95% by weight.

The dispersion preferably takes place after the chain extension. For this, the dissolved and chain-extended polyurethane polymer is optionally either introduced into the dispersion water with strong shear, such as, for example, with vigorous stirring, or, conversely, the dispersion water is stirred into the chain-extended polyurethane polymer solutions. Preferably, the water is added to the dissolved chain-extended polyurethane polymer.

The solvent still present in the dispersions after the dispersion step is then usually removed by distillation. Removal during dispersion is likewise possible.

The residual content of organic solvents in the polyurethane dispersions prepared in this way is typically less than 10% by weight, preferably less than 3% by weight, based on the total dispersion.

The pH of the aqueous polyurethane dispersions used according to the invention is typically less than 8.0, preferably less than 7.5 and is particularly preferably between 5.5 and 7.5.

The hair setting compositions within the context of the present invention can advantageously be present in the form of a spray, a mousse, a gel, an emulsion, a solution or a cream, such as in the form of a setting mousse, setting fluid, hairspray, styling gel, styling cream, aerosol mousse etc.

The hair setting composition according to the invention comprises preferably 0.1 to 20% by weight of the polyurethane described above and in particular 0.5 to 10% by weight, in each case based on the total weight of the composition.

Besides the polyurethane described above, the composition according to the invention can comprise further suitable film formers which can in particular also contribute to the setting and the styling of the hair.

The concentration of one or more further film formers can be from 0 to 20% by weight and in particular 0 to 10% by weight, in each case based on the total weight of the composition.

The film former or film formers are advantageously selected from the group of water-soluble or water-dispersible polyurethanes different from the polyurethanes used according to the invention, the polyureas, silicone resins and/or polyesters, and also the nonionic, anionic, amphoteric and/or cationic polymers and their mixtures.

Advantageous nonionic polymers which may be present in compositions according to the invention alone or in a mixture, preferably also with anionic and/or amphoteric and/or zwitterionic polymers, are selected from:
polyalkyloxazolines,
vinyl acetate homopolymers or copolymers. These include, for example, copolymers of vinyl acetate and acrylic acid esters, copolymers of vinyl acetate and ethylene, copolymers of vinyl acetate and maleic acid esters,
acrylic acid ester copolymers, such as, for example, the copolymers of alkyl acrylate and alkyl methacrylate, copolymers of alkyl acrylate and urethanes,
copolymers of acrylonitrile and nonionic monomer selected from butadiene and (meth)acrylate,
styrene homopolymers and copolymers. These include, for example, homopolystyrene, copolymers of styrene and alkyl(meth)acrylate, copolymers of styrene, alkyl methacrylate and alkyl acrylate, copolymers of styrene and butadiene, copolymers of styrene, butadiene and vinylpyridine,
polyamides,
vinyllactam homopolymers or copolymers, such as vinylpyrrolidone homo- or copolymers; these include, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate,
polysiloxanes,
homopolymers of N-vinylformamide e.g. PVF from National Starch.

Particularly preferred nonionic polymers are acrylic acid ester copolymers, homopolymers of vinylpyrrolidone and copolymers, polyvinylcaprolactam.

Very particularly preferred nonionic polymers are homopolymers of vinylpyrrolidone, e.g. Luviskol® K from BASF, copolymers of vinylpyrrolidone and vinyl acetate, e.g. Luviskol® VA grades from BASF or PVPVA® S630L from ISP, terpolymers of vinylpyrrolidone, vinyl acetate and propionate, such as, for example, Luviskol® VAP from BASF and polyvinylcaprolactams, e.g. Luviskol® PLUS from BASF.

Advantageous anionic polymers are homopolymers or copolymers with monomer units containing acid groups which are optionally copolymerized with comonomers which contain no acid groups. Suitable monomers are unsaturated, free-radically polymerizable compounds which have at least one acid group, in particular carboxylic acid, sulfonic acid or phosphonic acid.

Advantageous anionic polymers comprising carboxylic acid group are:
Acrylic acid or methacrylic acid homopolymer or copolymer or the salts thereof. These include, for example, the copolymers of acrylic acid and acrylamides and/or sodium salts thereof, copolymers of acrylic acid and/or methacrylic acid and an unsaturated monomer selected from ethylenes, styrene, vinyl esters, acrylic acid esters, methacrylic acid esters, optionally ethoxylated compounds, copolymers of vinylpyrrolidones, acrylic acid and C1-C20 alkyl methacrylates, e.g. Acrylidone® LM from ISP, copolymers of methacrylic acid, ethyl acrylates and tert-butyl acrylates, e.g. Luvimer® 100 P from BASF.
Crotonic acid derivative homopolymer or copolymer or the salts thereof. These include, for example, vinyl acetate/crotonic acid, vinyl acetate/acrylate and/or vinyl acetate/vinyl neodecanoate/crotonic acid copolymers, sodium acrylate/vinyl alcohol copolymers,
Unsaturated C4-C8 carboxylic acid derivatives or carboxylic acid anhydride copolymer selected from copolymers of maleic acid or maleic anhydride or fumaric acid or fumaric anhydride or itaconic acid or itaconic anhydride and at least one monomer selected from vinyl esters, vinyl ethers, vinyl halogen derivatives, phenyl vinyl derivatives, acrylic acid, acrylic acid esters or copolymers of maleic acid or maleic anhydride or fumaric acid or fumaric anhydride or itaconic acid or itaconic anhydride and at least one monomer selected from allyl esters, methallyl esters and optionally acrylamides, methacrylamides, alpha-olefin, acrylic acid esters, methacrylic acid esters, vinylpyrrolidones. Further preferred polymers are methyl vinyl ether/maleic acid copolymers, which are formed by hydrolysis of vinyl ether/maleic anhydride copolymers. These polymers can also be partially esterified (ethyl, isopropyl or butyl esters) or partially amidated.
Water-soluble or -dispersible anionic polyurethanes, e.g. Luviset® PUR from BASF, which are different from the polyurethanes according to the invention,
   where this list is of course not intended to be limiting.

Advantageous anionic polymers containing sulfonic acid group are salts of polyvinylsulfonic acid, salts of polystyrene sulfonic acid, such as, for example, sodium polystyrene sulfonate or salts of polyacrylamide sulfonic acid.

Particularly advantageous anionic polymers are acrylic acid copolymers, crotonic acid derivative copolymer, copolymers of maleic acid or maleic anhydride or fumaric acid or fumaric anhydride or itaconic acid or itaconic anhydride and at least one monomer selected from vinyl esters, vinyl ethers, vinyl halogen derivatives, phenyl vinyl derivatives, acrylic acid, acrylic acid esters and salts of polystyrene sulfonic acid.

Very particularly advantageous anionic polymers are acrylate copolymers, e.g. Luvimer from BASF, ethyl acrylate/N-tert-butylacrylamide/acrylic acid copolymers ULTRAHOLD® STRONG from BASF, VA/crotonate/vinyl neodecanoate copolymer, e.g. Resyn 28-2930 from National Starch, copolymers such as, for example, copolymers of methyl vinyl ether and maleic anhydride partially esterified e.g. GANTREZ® from ISP and sodium polystyrene sulfonates e.g. Flexan 130 from National Starch.

Advantageous amphoteric polymers can be selected from the polymers which contain units A and B distributed randomly in the polymer chain, where A means a unit which is derived from a monomer with at least one basic nitrogen atom, and B is a unit which originates from an acidic monomer which has one or more carboxy groups or sulfonic acid groups, or A and B can be groups which are derived from zwitterionic carboxybetaine monomers or sulfobetaine monomers; A and B can also be a cationic polymer chain which contains primary, secondary, tertiary or quaternary groups, in which at least one amino group carries a carboxy group or sulfonic acid group which is bonded via a hydrocarbon group, or B and C are part of a polymer chain with ethylene-α,β-dicarboxylic acid unit in which the carboxylic acid groups have been reacted with a polyamine which contains one or more primary or secondary amino groups.

Particularly advantageous amphoteric polymers are:
Polymers which are formed during the copolymerization of a monomer derived from a vinyl compound with carboxy group, such as, in particular, acrylic acid, methacrylic acid, maleic acid, α-chloroacrylic acid, and a basic monomer which is derived from a vinyl compound which is substituted and contains at least one basic atom, such as, in particular, dialkylaminoalkyl methacrylate and acrylate, dialkylaminoalkylmethacrylamide and -acrylamide. Such compounds have been described in the U.S. Pat. No. 3,836,537.
Polymers with units which are derived from: a) at least one monomer which is selected from the acrylamides or methacrylamides which are substituted on the nitrogen atom by an alkyl group, b) at least one acidic comonomer which contains one or more reactive carboxy groups, and c) at least one basic comonomer, such as esters of acrylic acid and methacrylic acid with primary, secondary, tertiary and quaternary amino substituents and the quartenization product of dimethylaminoethyl methacrylate with dimethyl sulphate or diethyl sulphate.
N-substituted acrylamides or methacrylamides particularly preferred according to the invention are compounds whose alkyl groups contain 2 to 12 carbon atoms, particularly N-ethylacrylamide, N-t-butylacrylamide, N-t-octylacrylamide, N-octylacrylamide, N-decylacrylamide, N-dodecylacrylamide, and the corresponding methacrylamides.
The acidic comonomers are selected in particular from acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.
Preferred basic comonomers are aminoethyl methacrylate, butylaminoethyl methacrylate, N,N-dimethylaminoethyl methacrylate, N-t-butylaminoethyl methacrylate.
Crosslinked and completely or partially acylated polyamino amides which are derived from polyamino amides of the following general formula:

—[CO—R—CO—Z]—
in which R is a divalent group which is derived from a saturated dicarboxylic acid, an aliphatic mono- or dicarboxylic acid with ethylenic double bond, an ester of these acids with a lower alkanol having 1 to 6 carbon atoms or a group which is formed upon the addition of one of these acids onto a bis-primary or bis-secondary amine, and Z is a group which is derived from a bis-primary, mono- or bis-secondary polyalkylenepolyamine, and preferably: a) in quantitative fractions from 60 to 100 mol % the groups —NH—[(CH₂)ₓ—NH—]ₚ— where x=2 and p=2 or 3 or x=3 and p=2, where this group is derived from diethylenetriamine, triethylenetetramine or dipropylenetriamine; b) in quantitative fractions of from 0 to 40 mol % the group —NH—[(CH₂)ₓ—NH—]ₚ—, in which x=2 and p=1, which is derived from ethylenediamine, or the group which originates from piperazine:

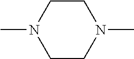

c) in quantitative fractions of from 0 to 20 mol %, the group —H—(CH₂)₆—NH—, which is derived from hexamethylenediamine, where these polyaminoamides are crosslinked by adding a bifunctional crosslinking agent, which is selected from epihalohydrins, diepoxides, dianhydrides and bis-unsaturated derivatives, in an amount of from 0.025 to 0.35 mol of crosslinking agent per amino group of the polyaminoamide, and acylated with acrylic acid, chloroacetic acid or an alkanesultone or salts thereof.

The saturated carboxylic acids are preferably selected from the acids having 6 to 10 carbon atoms, such as adipic acid, 2,2,4-trimethyladipic acid and 2,4,4,-trimethyladipic acid, terephthalic acid; acids with ethylenic double bond, such as, for example, acrylic acid, methacrylic acid and itaconic acid.

The alkanesultones used in the acylation are preferably propanesultone or butanesultone, the salts of the acylating agents are preferably the sodium salts or potassium salts.
Polymers with zwitterionic units of the following formula:

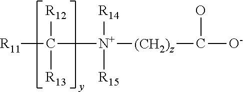

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group which is selected such that the sum of the carbon atoms R₁₄ and R₁₅ does not exceed 10.
Polymers which contain such units can also have units which originate from non-zwitterionic monomers, such as dimethyl- and diethylaminoethyl acrylate or dimethyl- and diethylaminoethyl methacrylate or alkyl acrylates or alkyl methacrylates, acrylamides or methacrylamides or vinyl acetate.
Polymers which are derived from chitosan and contain monomer units which correspond to the following formulae:

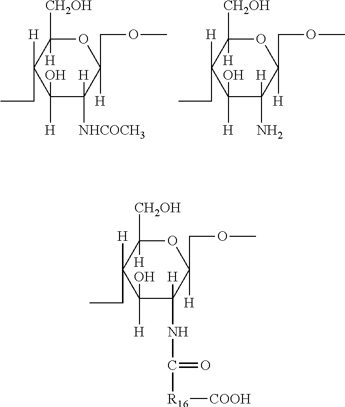

where the first unit is present in quantitative fractions of from 0 to 30%, the second unit is present in quantitative fractions of from 5 to 50% and the third unit is present in quantitative fractions of from 30 to 90%, with the proviso that, in the third unit, R₁₆ is a group of the following formula:

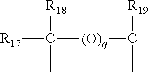

in which: if q=0, the groups R₁₇, R₁₈ and R₁₉, which are identical or different, are in each case a hydrogen atom, methyl, hydroxy, acetoxy or amino, a monoalkylamine radical or a dialkylamine radical which is optionally interrupted by one or more nitrogen atoms and/or optionally one or more of the groups amino, hydroxy, carboxy, alkylthio, sulfonic acid, alkylthio, whose alkyl group carries an amino radical, where at least one of the groups R₁₇, R₁₈ and R₁₉ is in this case a hydrogen atom; or if q=1, the groups R₁₇, R₁₈ and R₁₉ are in each case a hydrogen atom, and also the salts which form these compounds with bases or acids.
Polymers which correspond to the following general formula and which are described, for example, in the French patent 1 400 366:

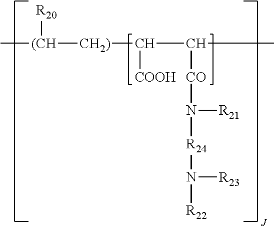

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl or a group of the formula: —R₂₄—N(R₂₂)₂, where R₂₄ is a group —CH₂—CH₂, —CH₂—CH₂—CH₂— or —CH₂—CH(CH₃)— and where R₂₂ has the meanings given above.
Polymers which can be formed during the N-carboxyalkylation of chitosan, such as N-carboxymethyl chitosan or N-carboxybutyl chitosan.
Amphoteric polymers of the type -D-X-D-X, which are selected from:
a) polymers which are formed through the action of chloroacetic acid or sodium chloroacetate on compounds with at least one unit of the following formula: D-X-D-X,
   in which D is the group

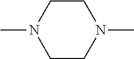

and X is the symbols E or E′, where E or E′, which are identical or different, are a divalent group, which is a straight-chain or branched alkylene group having up to 7 carbon atoms in the main chain, which is present in unsubstituted form or is substituted by hydroxy groups and can contain one or more oxygen atoms, nitrogen atoms or sulphur atoms and 1 to 3 aromatic and/or heterocyclic rings; where the oxygen atoms, nitrogen atoms and sulphur atoms are present in the form of the following groups: ether, thioether, sulfoxide, sulfone, sulfonium, alkylamine, alkenylamine, hydroxy, benzylamine, amine oxide, quaternary ammonium, amide, imide, alcohol, ester and/or urethane.
b) Polymers of the formula D-X-D-X, in which D is the group

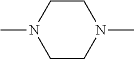

and X is the symbol E or E′ and at least once E′; where E has the meanings given above and E′ is a divalent group, which is a straight-chain or branched alkylene group having up to 7 carbon atoms in the main chain, which is present in unsubstituted form or is substituted by one or more hydroxy groups and contains one or more nitrogen atoms, where the nitrogen atom is substituted by an alkyl group, which is optionally interrupted by an oxygen atom and obligatorily contains one or more carboxy functions or one or more hydroxy functions and is betainized through reaction with chloroacetic acid or sodium chloroacetate.
Alkyl(C₁₋₅) vinyl ether/maleic anhydride copolymers which are partially modified by semiamidation with an N,N-dialkylaminoalkylamine, such as N,N-dimethylaminopropylamine or an N,N-dialkylaminoalcohol. These polymers can also contain further comonomers, such as vinylcaprolactam.

Very particularly advantageous amphoteric polymers are, for example, the copolymers octylacrylamide/acrylates/butylaminoethyl methacrylate copolymers which are commercially available under the names AMPHOMER®, AMPHOMER® LV 71 or BALANCE® 47 from NATIONAL STARCH, and methyl methacrylate/methyl dimethylcarboxymethylammonium ethyl methacrylate copolymers.

It is optionally advantageous to neutralize the anionic and amphoteric polymers using suitable bases in order to improve their solubility and/or dispersibility in water.

The following bases can be used as neutralizing agents for polymers which contain acid groups: hydroxides whose cation is an ammonium or an alkali metal, such as, for example, NaOH or KOH.

Other neutralizing agents are primary, secondary or tertiary amines, amino alcohols or ammonia. Preference is given here to 2-amino-2-methyl-1,3-propanediol (AMPD), 2-amino-2-ethyl-1,3-propanediol (AEPD), 2-amino-2-methyl-1-propanol (AMP), 2-amino-1-butanol (AB), 2-amino-1,3-propanediol, monoethanolamine (MEA), diethanolamine (DEA), triethanolamine (TEA), monoisopropanolamine (MIPA), diisopropanolamine (DIPA), triisopropanolamine (TIPA), dimethyllaurylamine (DML), dimethylmyristalamine (DMM) and dimethylstearamine (DMS).

The neutralization can be partial or complete depending on the intended application.

In some cases, it is also possible, but less preferred, to use cationic polymers, such as, for example, polymers which contain primary, secondary, tertiary and/or quaternary amino groups which are bonded as part of the polymer chain or directly to the polymer chain.

The cosmetic acceptable medium comprises in particular water and optionally a cosmetically suitable solvent. The preferred solvents are aliphatic alcohols having C2-4 carbon atoms, such as ethanol, isopropanol, t-butanol, n-butanol; polyol, such as propylene glycol, glycerol, ethylene glycol and polyol ethers; acetone; unbranched or branched hydrocarbons, such as pentane, hexane, isopentane and cyclic hydrocarbons, such as cyclopentane and cyclohexane; and mixtures thereof.

A very particularly preferred solvent is ethanol.

However, the content of such solvents, in accordance with the fact that according to the invention these are preferably low-VOC hair setting compositions, is preferably less than 80% by weight, or preferably less than 55% by weight, yet more preferably less than 40% by weight.

The water fraction can be in particular in the range, for example, from 20 to 94% by weight, preferably from 30 to 80% by weight, more preferably from more than 45 to 70% by weight, based on the total weight of the composition. The medium is advantageously an aqueous-alcoholic mixture. The quantitative fraction of the alcohol in the mixture is in the range from 0 to 90% by weight, preferably 0 to 70% by weight, more preferably 0 to 55% by weight, yet more preferably 0 to 40% by weight, based on the total weight of the composition.

The hair setting compositions according to the invention can furthermore advantageously comprise thickeners. Advantageous thickeners are:
Crosslinked or uncrosslinked acrylic acid or methacrylic acid homopolymers or copolymers. These include crosslinked homopolymers of methacrylic acid or acrylic acid, copolymers of acrylic acid and/or methacrylic acid and monomers which are derived from other acrylic or vinyl monomers, such as C10-30 alkyl acrylates, C10-30-alkyl methacrylates and vinyl acetate.
Thickening polymers of natural origin, for example based on cellulose, guar gum, xanthan, scleroglucan, gellan gum, rhamsan and karaya gum, alginates, maltodextrin, starch and its derivatives, carob seed flour, hyaluronic acid.
Nonionic, anionic, cationic or amphoteric associative polymers, e.g. based on polyethylene glycols and their derivatives, or polyurethanes.
Crosslinked or uncrosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homopolymers or copolymers and crosslinked copolymers of 2-acrylamido-2-methylpropanesulfonic acid.

Very particularly advantageous thickeners are xanthan gum, such as the products supplied under the names Keltrol® and Kelza® by CP Kelco or the products from RHODIA with the name Rhodopol, and guar gum, such as the products available under the name Jaguar® HP105 from RHODIA.

Very particularly advantageous thickeners are crosslinked homopolymers of methacrylic acid or acrylic acid which are commercially available from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS, and from PROTEX under the names Modarez® V 1250 PX, Modarez® V2000 PX, Viscaron® A1600 PE and Viscaron® A700 PE.

Very particularly advantageous thickeners are crosslinked polymers of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are commercially available, for example, from Lubrizol under the names Carbopol® 1342, Carbopol® 1382, Pemulen® TR1 or Pemulen® TR2 and from ISP under the names Ultrathix P-100 (INCI: Acrylic Acid/VP Crosspolymer).

Very particularly advantageous thickeners are crosslinked copolymers of 2-acrylamido-2-methylpropanesulfonic acid. Such copolymers are available, for example, from Clariant under the names Aristoflex® AVC (INCI: Ammonium Acryloyldimethyltaurate/VP Copolymer).

If the thickeners are used, they are generally present in a concentration of from 0% to 2% by weight, preferably 0% to 1% by weight, based on the total weight of the composition.

The hair setting compositions according to the invention can moreover comprise a propellant gas. Here, it is advantageous to use the propellant gas in an amount of from 0 to 40% by weight and particularly preferably in a concentration of from 0 to 20% by weight, based on the total weight of the formulation.

The propellant gases preferred according to the invention are hydrocarbons such as propane, isobutane and n-butane, and mixtures thereof. However, compressed air, carbon dioxide, nitrogen, nitrogen dioxide and dimethyl ether, and mixtures of all of these gases can also be used advantageously according to the invention.

The person skilled in the art is of course aware that there are propellant gases which are nontoxic per se which would in principle be suitable for realizing the present invention in the form of aerosol preparations, but which nevertheless have to be dispensed with on account of an unacceptable impact on the environment or other accompanying circumstances, in particular fluorocarbons and chlorofluorocarbons (CFCs), such as, for example, 1,2-difluoroethane (propellant 152 A).

Furthermore, hair care active ingredients can be used in the hair setting formulations according to the invention. Care substances which can preferably be used are cyclic polydimethylsiloxanes (cyclomethicones) in concentrations of e.g. 0-1.0% by weight of the total formulation or silicone surfactants (polyether-modified siloxanes) of the type dimethicone copolyol or simethicone, e.g. in concentrations of 0-1.0% by weight of the total weight of the composition. Cyclomethicones are supplied, inter alia, under the trade names Abil® K4 by Goldschmidt or e.g. DC 244, DC 245 or DC 345 by Dow Corning. Dimethicone copolyols are supplied, for example, under the trade name DC 193 by Dow Corning or Belsil® DM 6031 by Wacker.

Optionally, conventional additives may likewise be present in the hair setting composition, for example in order to impart certain modifying properties to the composition; these are silicones or silicone derivatives, wetting agents, humectants, softeners such as glycerol, glycol and phthalic esters and ethers, fragrances and perfumes, UV absorbers, dyes, pigments, and other colorants, anticorrosive agents, neutralizing agents, antioxidants, antiadhesives, combining agents and conditioners, antistatic agents, lustre agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlizing agents, aesthetic enhancers, fatty acids, fatty alcohols, triglycerides, botanic extracts, clarifying auxiliaries and film formers.

These additives are generally present in a concentration of from about 0.001% to 15% by weight, preferably 0.01% to 10% by weight, based on the total weight of the hair setting composition.

The hair setting compositions according to the invention can advantageously be in the form of a pump spray or aerosol packaging. The hair setting compositions according to the invention can advantageously be foamed using a propellant gas. Accordingly, pump spray, aerosol packagings and foam dispensers based on pump spray or aerosol packaging which contain the hair setting composition according to the invention are likewise a constituent of the invention.

A preferred embodiment of the hair setting compositions according to the invention is in the form of a spray, which additionally comprises one or more of the following constituents: cosmetically suitable solvents, such as aliphatic alcohols having 2-4 carbon atoms, preferably ethanol, polyols, acetone, unbranched or branched hydrocarbons, cyclic hydrocarbons and mixtures thereof, and also propellant gases, such as hydrocarbons, compressed air, carbon dioxide, nitrogen, nitrogen dioxide, dimethyl ether, fluorocarbons and chlorofluorocarbons, preferably dimethyl ether and/or a propane/butane mixture.

The present invention is illustrated by reference to the examples below, which are not to be understood as being limiting. Unless stated otherwise, all quantitative data, fractions and percentages are based on the weight and the total amount or on the total weight of the compositions.

### EXAMPLES

Unless designated otherwise, all of the percentages refer to the weight.

Unless noted differently, all of the analytical measurements refer to measurements at temperatures of 23° C.

The solids or solid-body contents are determined by heating a weighed sample at 125° C. to constant weight. At constant weight, the solid-body content is calculated by reweighing the sample.

Unless expressly mentioned otherwise, NCO contents were determined volumetrically in accordance with DIN-EN ISO 11909.

The control on free NCO groups was carried out by means of IR spectroscopy (band at 2260 cm⁻¹).

The stated viscosities were determined by means of rotary viscometry in accordance with DIN 53019 at 23° C. using a rotary viscometer from Anton Paar Germany GmbH, Ostfildern, Germany.

The average particle sizes (the number-average is stated) of the polyurethane dispersions were determined following dilution with deionized water by means of laser correlation spectroscopy (instrument: Malvern Zetasizer 1000, Malvern Inst. Limited).

### Substances Used and Abbreviations:

Diaminosulfonate: NH₂—CH₂CH₂—NH—CH₂CH₂—SO₃Na (45% strength in water)
Desmophen® 2020/C2200: Polycarbonate polyol, OH number 56 mg of KOH/g, number-average molecular weight 2000 g/mol (Bayer MaterialScience AG, Leverkusen, Germany)
PolyTHF® 2000: Polytetramethylene glycol polyol, OH number 56 mg of KOH/g, number-average molecular weight 2000 g/mol (Basf Ag, Ludwigshafen, Germany)
PolyTHF® 1000: Polytetramethylene glycol polyol, OH number 112 mg of KOH/g, number-average molecular weight 1000 g/mol (Basf Ag, Ludwigshafen, Germany)
Polyether LB 25: Monofunctional polyether based on ethylene oxide/propylene oxide of number-average molecular weight 2250 g/mol, OH number 25 mg of KOH/g (Bayer MaterialScience AG, Leverkusen, Germany)

### Example 1

### Polyurethane Dispersion 1

987.0 g of PolyTHF® 2000 (component A2)), 375.4 g of PolyTHF® 1000 (component A2)), 761.3 g of Desmophen® C2200 (component A2)) and 44.3 g of Polyether LB 25 (component A4)) were heated to 70° C. in a standard stirring apparatus. A mixture of 237.0 g of hexamethylene diisocyanate (component A1)) and 313.2 g of isophorone diisocyanate (component A1)) was then added and the mixture was stirred at 120° C. until the theoretical NCO value was reached. The finished prepolymer was dissolved with 4830 g of acetone and in so doing cooled to 50° C., and then a solution of 25.1 g of ethylenediamine (component B1)), 116.5 g of isophoronediamine (component B1)), 61.7 g of diaminosulfonate (component B2)) and 1030 g of water was then metered in. The afterstirring time was 10 min. The mixture was then dispersed by adding 1250 g of water. The solvent was removed by distillation in vacuo.

The resulting white dispersion had the following properties:

| | |
|---|---|
| Solids content: | 61% |
| Particle size (LCS): | 312 nm |
| Viscosity (viscometer: 23° C.): | 241 mPas |
| pH (23° C.): | 6.02 |
| pH (23° C.): | 7.15 |

### Example 2

### Polyurethane Dispersion 2

450 g of PolyTHF® 1000 (component A2)) and 2100 g of PolyTHF® 2000 (component A2)) were heated to 70° C. A mixture of 225.8 g of hexamethylene diisocyanate (component A1)) and 298.4 g of isophorone diisocyanate (component A1)) was then added and the mixture was stirred at 100-115° C. until the actual NCO value fell below the theoretical NCO value. The finished prepolymer was dissolved with 5460 g of acetone at 50° C. and then a solution of 29.5 g of ethylenediamine (component B1)), 143.2 g of diaminosulfonate (component B2)) and 610 g of water was then metered in. The afterstirring time was 15 min. The mixture was then dispersed by adding 1880 g of water. The solvent was removed by distillation in vacuo and a storage-stable dispersion was obtained.

| | |
|---|---|
| Solids content: | 56% |
| Particle size (LCS): | 276 nm |
| Viscosity: | 1000 mPas |

### Example 3

### Polyurethane Dispersion 3

1649.0 g of a polyester of adipic acid, hexanediol and neopentyl glycol with an average molecular weight of 1700 g/mol (component A2)) were heated to 65° C. 291.7 g of hexamethylene diisocyanate (component A1)) were then added and the mixture was stirred at 100-115° C. until the actual NCO value fell below the theoretical NCO value. The finished prepolymer was dissolved with 3450 g of acetone at 50° C. and then a solution of 16.8 g of ethylenediamine (component B1)), 109.7 g of diaminosulfonate (component B2)) and 425 g of water was metered in. The afterstirring time was 15 min. The mixture was then dispersed by adding 1880 g of water. The solvent was removed by distillation in vacuo and a storage-stable dispersion was obtained.

| | |
|---|---|
| Solids content: | 42% |
| Particle size (LCS): | 168 nm |
| Viscosity: | 425 mPas |
| pH: | 7.07 |

### Example 4

### Polyurethane Dispersion 4

340 g of a polyester of adipic acid, hexanediol and neopentyl glycol with an average molecular weight of 1700 g/mol (component A2)) were heated to 65° C. 60.1 g of hexamethylene diisocyanate (component A1)) were then added and the mixture was stirred at 105° C. until the actual NCO value fell below the theoretical NCO value. The finished prepolymer was dissolved with 711 g of acetone at 50° C. and then a solution of 2.1 g of ethylenediamine (component B1)), 32.4 g of diaminosulfonate (component B2)) and 104.3 g of water was metered in. The afterstirring time was 15 min. The mixture was then dispersed by adding 1880 g of water. The solvent was removed by distillation in vacuo, and a storage-stable dispersion was obtained.

| | |
|---|---|
| Solids content: | 40% |
| Particle size (LCS): | 198 nm |
| Viscosity: | 700 mPas |
| pH: | 6.31 |

### Example 5

### Polyurethane Dispersion 5

450 g of PolyTHF® 1000 (component A2)) and 2100 g of PolyTHF® 2000 (component A2)) were heated to 70° C. A mixture of 225.8 g of hexamethylene diisocyanate (component A1)) and 298.4 g of isophorone diisocyanate (component A1)) was then added and the mixture was stirred at 100-115° C. until the actual NCO value fell below the theoretical NCO value. The finished prepolymer was dissolved with 5460 g of acetone at 50° C. and then a solution of 351 g of diaminosulfonate (component B2)) and 610 g of water was metered in. The afterstirring time was 15 min. The mixture was then dispersed by adding 1880 g of water. The solvent was removed by distillation in vacuo and a storage-stable dispersion was obtained.

| | |
|---|---|
| Solids content: | 42% |
| Viscosity: | 1370 mPas |

### Application-Related Comparative Experiments:

For the so-called “Curl Retention” experiments, commercial European mixed hair from Kerling (useful length: 19 cm, Kerling colour number 6/0) are used. The hair is subjected to a standardized washing procedure prior to use. The hair softened for 15 min in water is shampooed for 2 minutes using 15% by weight sodium dodecyl sulphate solution, thoroughly rinsed with warm water, blow-dried on a cold setting and conditioned at 22° C. and 55% relative humidity. Hair tresses 0.5 cm in width are wound whilst damp onto spiral rollers, blow-dried on a hot setting for 35 min, then sprayed with polymer dispersion and conditioned overnight.

For the application of the polymer dispersion, a spray head from Seaquist Perfect Dispensing GmbH is used: pump atomizer type PZ1/150 HV (24/410). From a distance of 30 cm, the compositions according to the invention are applied to the hair material by brisk actuation of the spray head.

The “Curl Retention” experiment is carried out in a special climatized chamber with a relative humidity of >98%. The temperature of the chamber is 30° C. The prepared tresses are hung simultaneously in the chamber. The length of the curls is read off on a scale at certain times. Each experiment is carried out on three tresses.

| Experiment | A | B |
|---|---|---|
| Ethanol (% by wt.) | 50 | 50 |
| Polyurethane (% by wt. solid)¹⁾ | 4 | 2 |
| Water (% by wt.) | ad 100 | ad 100 |
| Spray bursts | 12 | 4 |

Parts by weight are stated.
¹⁾Based on solid in the aqueous polyurethane dispersion.

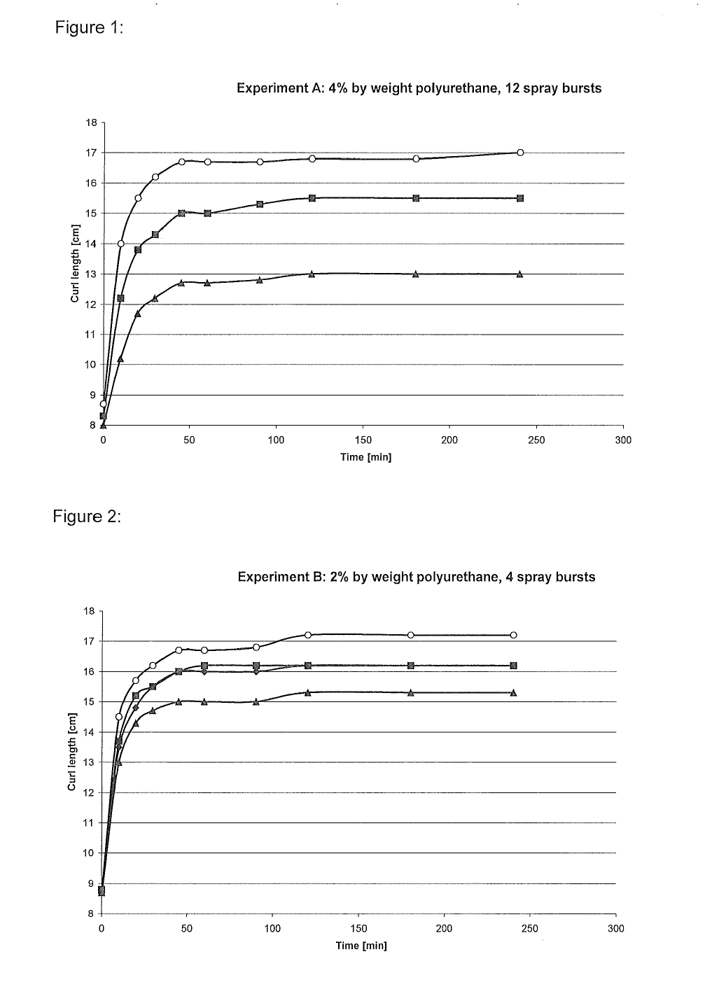

FIG. 1 shows the results achieved in experiment A.

FIG. 2 shows the results achieved in experiment B.

In the Figures:

O means blank experiments, without polyurethane

### Polyurethanes of the Prior Art, Comprising Dimethylpropanoic Acid:

(Polyurethanes comprising dimethylolpropanoic acid are usually prepared by incorporating the dimethylolpropanoic acid into a prepolymer which is water-soluble or water-dispersible, cf. WO 94/03510, EP 0619111 and EP 637600).

▪ Polyurethane of the prior art, Luviset® PUR from BASF, INCI name: Polyurethane-1, polyurethane of a diisocyanate, dimethylolpropanoic acid, polyesterdiol and aliphatic diol.

♦ Polyurethane of the prior art, DynamX® POLYMER from National Starch, INCI name: Polyurethane 14 (and) AMP-acrylates copolymer, polyurethane-14 consists of isophorone diisocyanate, propyleneglycol, dimethylpropanoic acid, diol, polyether.

▴ Polyurethane according to the invention as in example 4

The experiments clearly show that the best curl hold is achieved with the polyurethane used according to the invention.

### Application-Related Examples:

(Parts by weight are stated).

“Pump-setting spray”

| | A | B |
|---|---|---|
| Polyurethane according to the | 2 | 10 |
| invention (based on solid) | | |
| Ethanol | 55 | 30 |
| Perfume | q.s. | q.s. |
| Water | ad 100 | ad 100 |

Aerosol hairsprays

| | C | D | E | F | G |
|---|---|---|---|---|---|
| Polyurethane according to the | 5 | 10 | 2 | 5 | 8 |
| invention (based on solid) | | | | | |
| Octylacrylamide/acrylate/ | | | | 1.8 | |
| butylaminoethyl methacrylate¹ | | | | | |
| (based on solid) | | | | | |
| Acrylate copolymer² | | | 2 | | |
| Aminomethylpropanol | | | q.s | q.s | |
| Glycerol | | 0.5 | | | |
| Panthenol | | | 0.5 | | 0.5 |
| PEG/PPG-18/18 dimethicone | | | | 0.5 | |
| PEG-12 dimethicone | | 0.05 | | | |
| Propylene glycol | | | | 0.5 | |
| Cyclomethicone | | | 1.0 | | 1.0 |
| Benzophenone-3 | | 0.1 | 0.1 | | 0.1 |
| Perfume | q.s | q.s | q.s | q.s | q.s |
| Ethanol | 14.5 | 20 | 60 | 30 | 20 |
| Water | ad 100 | ad 100 | ad 100 | ad 100 | ad 100 |
| Propane/butane 3.5 bar (20° C.) | | | 20 | 10 | |
| Dimethyl ether | 40 | 30 | | 30 | 20 |
| Fluorocarbon 152 A | | | | | 20 |

Hair mousse

| | H | I |
|---|---|---|
| Polyurethane according to the | 2 | 4 |
| invention (based on solid) | | |
| Glycerol | 0.1 | |
| Panthenol | 0.05 | 0.5 |
| Polyquaternium-4 | 2 | |
| Cetyltrimethylammonium chloride | 0.2 | 0.5 |
| PEG-12 dimethicone | | 0.5 |
| Cyclomethicone | | 0.5 |
| Benzophenone-3 | 0.1 | |
| Perfume | q.s. | q.s. |
| Ethanol | 15 | 10 |
| Water | ad 100 | ad 100 |
| Preservative | q.s. | q.s. |
| Dimethyl ether | 10 | 7 |
| Fluorocarbon 152 A | | 3 |

Hair gel/cream

| | J | K | L |
|---|---|---|---|
| Polyurethane according to the | 5 | 2 | 8 |
| invention (based on solid) | | | |
| Carbomer | 0.8 | | |
| Acrylic acid/VP copolymer | | 0.5 | |
| Ammonium | | | 0.8 |
| acryloyldimethyltaurate/VP copolymer | | | |
| Glycerol | 0.5 | | |
| Panthenol | | 0.5 | 0.5 |
| Propylene glycol | | | 0.2 |
| Cyclomethicone | | 0.2 | |
| Neutralizing agent | q.s. | q.s. | q.s. |
| Perfume | q.s. | q.s. | q.s. |
| Ethanol | 20 | | |
| Water | ad 100 | ad 100 | ad 100 |
| Preservative | q.s. | q.s. | q.s. |

¹Amphomer, National starch
²Luvimer P-100, BASF

1-14. (canceled)

15. Hair setting composition comprising at least one polyurethane obtainable by reacting one or more water-insoluble, non-water-dispersible, isocyanate-functional polyurethane prepolymers A) with one or more amino-functional compounds B).

16. Hair setting composition comprising at least one polyurethane obtainable by reacting one or more isocyanate-functional polyurethane prepolymers A), which essentially have neither ionic nor ionogenic groups, with one or more amino-functional compounds B).

17. Hair setting composition according to claim 15, wherein the amino-functional compounds B) are selected from primary and/or secondary amines and/or diamines.

18. Hair setting composition according to claim 15, wherein the amino-functional compounds B) include at least one diamine.

19. Hair setting composition according to claim 15, wherein the amino-functional compounds B) are selected from amino-functional compounds B2), which have ionic and/or ionogenic groups, and amino-functional compounds B1), which have no ionic and/or ionogenic groups.

20. Hair setting composition according to claim 15, wherein the amino-functional compounds B) include at least one amino-functional compound B2) which has ionic and/or ionogenic groups, preferably 2-(2-aminoethylamino)ethane sulfonic acid and/or salts thereof.

21. Hair setting composition according to claim 15, wherein the amino-functional compounds B) include at least one amino-functional compound B1) that have no ionic and/or ionogenic groups, preferably a diamine which has no ionic and/or ionogenic groups.

22. Hair setting composition according to claim 15, wherein the amino-functional compounds B) include both amino-functional compounds B2) which have ionic and/or ionogenic groups, and also amino-functional compounds B1) which have no ionic and/or ionogenic group.

23. Hair setting composition according to claim 15, wherein the prepolymers A) are obtainable by reacting one or more polyols selected from the group which consists of polyether polyols, polycarbonate polyols, polyether-polycarbonate polyols and/or polyester polyols, and one or more polyisocyanates.

24. Hair setting composition according to claim 15, wherein the polyurethane contains at least one sulfonic acid and/or sulfonate group, preferably a sodium sulfonate group.

25. Hair setting composition according to claim 15, characterized in that it has a content of volatile organic compounds (VOC) of less than 80% by weight, based on the hair setting composition.

26. Method of shaping or setting the hair, which involves applying a cosmetic composition, comprising at least one polyurethane obtainable by reacting one or more water-insoluble, non-water-dispersible, isocyanate-functional polyurethane prepolymers A) with one or more amino-functional compounds B), to the hair.
